(19) United States
(12) Patent Application Publication
(10) Pub. No.: US 2023/0064879 A1
(43) Pub. Date: Mar. 2, 2023

(54) NANODRUG PARTICLES, THE USE THEREOF, AND PREPARATION METHOD THEREOF

(71) Applicant: NATIONAL YANG MING CHIAO TUNG UNIVERSITY, Hsinchu City (TW)

(72) Inventors: Dean-Mo LIU, Hsinchu City (TW); Yung-Hsin CHANG, Taoyuan City (TW); Yu-Wen CHEN, Hsinchu County (TW)

(21) Appl. No.: 17/589,223

(22) Filed: Jan. 31, 2022

(30) Foreign Application Priority Data
Aug. 19, 2021 (TW) 110130767

(51) Int. Cl. A61K 9/127; A61K 31/4745; A61K 9/51

(52) U.S. Cl. CPC A61K 9/1273; A61K 31/4745; A61K 9/5161; B82Y 5/00

(57) ABSTRACT

A nanodrug particle includes alginate and a camptothecin compound. The camptothecin compound is grafted onto the alginate, and the alginate and the camptothecin compound self-assemble and form a nanosphere. The disclosure also provides a method for preparing a nanodrug particle; the method includes: modifying alginate to form alginate having amine groups; modifying a camptothecin compound to form a camptothecin compound having a carboxyl group; forming a camptothecin-alginate polymer by reacting the alginate having amine groups with the camptothecin compound having a carboxyl group, wherein the camptothecin-alginate polymer self-assembles in an aqueous solution and forms a nanosphere.

### CROSS-REFERENCE TO RELATED APPLICATION

This application claims priority to Taiwan Application Serial Number 110130767, filed Aug. 19, 2021, which is herein incorporated by reference in its entirety.

### BACKGROUND

### Field of Invention

The present disclosure relates to nanodrug particles formed by modifying a camptothecin compound and the preparation method for the same.

### Description of Related Art

Camptothecin (CPT) is a topoisomerase inhibitor found in the bark and the stem of genus Camptotheca. Camptothecin has shown excellent anti-cancer effects against various cancers in preclinical stage; however, it is difficult to use camptothecin due to its low solubility. Camptothecin derivatives, such as Topotecan and Irinotecan, have therapeutic effects on breast cancer, small cell cancer, colorectal cancer, etc., but their clinical application is often limited because of poor water solubility or low bioavailability and serious side effects.

### SUMMARY

Some embodiments of the present disclosure provide a nanodrug particle comprising: alginate and a camptothecin compound. The camptothecin compound is grafted onto the alginate. The alginate and the camptothecin compound self-assemble and form a nanosphere.

In some embodiments, in the nanodrug particle, the camptothecin compound is grafted onto the alginate through

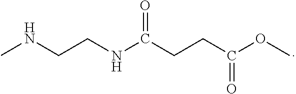

In some embodiments, in the nanodrug particle, the linkage segment between the camptothecin compound and the alginate comprises an amide group.

In some embodiments, in the nanodrug particle, the molecular weight of the alginate is less than 40,000 Da.

In some embodiments, in the nanodrug particle, the camptothecin compound is selected from the group consisting of camptothecin, Topotecan, Irinotecan, and SN-38.

In some embodiments, the nanodrug particle has a particle diameter ranging from about 200 nm to about 600 nm.

In some embodiments, the nanodrug particle is a micelle, and the outer portion of the micelle is a hydrophilic layer composed of the alginate, and the interior portion of the micelle is a hydrophobic layer composed of the camptothecin compound.

In some embodiments, the nanodrug particle further comprises a hydrophobic molecule dissolved in the hydrophobic layer of the micelle. In some embodiments, in the nanodrug particle, the hydrophobic molecule is an anti-cancer drug or a contrast agent.

Some embodiments of the present disclosure provide the use of the nanodrug particle for the manufacture of an anti-cancer drug.

Some embodiments of the present disclosure provide a treatment method for cancer, including: administering a nanodrug particle to a cancer patient. The nanodrug particle comprises alginate and a camptothecin compound. The camptothecin compound is grafted onto the alginate, and the alginate and the camptothecin compound self-assemble and form a nanosphere.

Some embodiments of the present disclosure provide a method for preparing a nanodrug particle, including: modifying alginate to form alginate having amine groups (—NH₂); modifying a camptothecin compound to form a camptothecin compound having a carboxyl group (—COOH); reacting the alginate having amine groups with the camptothecin compound having a carboxyl group to form a camptothecin-alginate polymer, wherein the camptothecin-alginate polymer self assembles in an aqueous solution and forms a nanosphere.

In some embodiments, the method for preparing the nanodrug particle further comprises: before the step of modifying the alginate, the alginate is degraded until the molecular weight of the alginate is less than about 40,000 Da.

In some embodiments, in the method for preparing the nanodrug particle, the step of modifying the alginate comprises using ethylenediamine as a reactant.

In some embodiments, in the method for preparing the nanodrug particle, the step of modifying the camptothecin compound comprises using succinic anhydride as a reactant.

In some embodiments, in the method for preparing the nanodrug particle, the camptothecin compound is selected from the group consisting of camptothecin, Topotecan, Irinotecan, and SN-38.

In some embodiments, the method for preparing the nanodrug particle further comprises: adding a hydrophobic compound, and mixing the hydrophobic compound with the nanosphere so that the hydrophobic compound is dissolved in the interior portion of the nanosphere.

In some embodiments, in the method for preparing the nanodrug particle, the hydrophobic compound is an anti-cancer drug or a contrast agent.

### BRIEF DESCRIPTION OF THE DRAWINGS

FIG. 1A is a schematic diagram of a method for forming a camptothecin-alginate polymer.

FIG. 1B illustrates a schematic diagram of a self-assembled nanosphere formed from the camptothecin-alginate polymer.

FIG. 10 illustrates a nanosphere according to some embodiments of the present disclosure.

FIG. 2A illustrates a schematic diagram of a step for modifying camptothecin to form camptothecin having a carboxyl group (—COOH).

FIG. 2B illustrates a schematic diagram of a step for modifying alginate to form alginate having amine groups (—NH₂).

FIG. 2C illustrates a schematic diagram of a step for reacting the alginate having amine groups with the camptothecin having a carboxyl group to form a camptothecin-alginate polymer.

FIG. 3 shows a UV-VIS absorption light spectrum of the camptothecin having a carboxyl group, in accordance with an example.

FIG. 4 is a graph showing the particle diameter size distribution of the nanospheres according to an example.

FIG. 5 is a graph showing the measurement results of the zeta potential of the nanospheres according to an example.

FIG. 6A is a graph showing the critical micelle concentrations of the nanospheres formed from grafting camptothecin onto alginate according to an example, and the critical micelle concentrations were measured by using Nile Red as a probe.

FIG. 6B is a graph showing the relationship between the Log value of the concentration of the nanospheres and the fluorescence intensity, in accordance with the example of FIG. 6A.

FIGS. 7A to 7C show the electron microscope images of the nanospheres according to some examples.

FIG. 8 is a graph showing the in vitro drug release results of the camptothecin and the camptothecin-alginate polymer according to an example.

FIG. 9A is a graph showing the results of the cytotoxicity assay on A549 cells according to an example.

FIG. 9B is a graph showing the results of the cytotoxicity assay on HT-29 cells according to an example.

### DETAILED DESCRIPTION

In order to make the description of the present disclosure more detailed and complete, the embodiments and specific examples of the present disclosure will be described below with reference to the accompanying drawings; but this is not the only way to implement or use the specific embodiments of the present disclosure. The embodiments disclosed below can be combined or replaced with each other under beneficial circumstances, and other embodiments can be added to an embodiment without further description or explanation.

The structural formula of camptothecin (CPT) is:

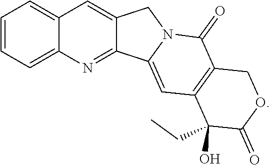 [SMILES: CC[C@@]1(O)C(=O)OCc2c1cc1n(c2=O)Cc2cc3ccccc3nc2-1]

In order to improve the water solubility of camptothecin and enhance the drug delivery properties of camptothecin, some embodiments of the present disclosure provide a method for grafting camptothecin onto aminated alginate and also provide a nanosphere formed from self-assembly of a camptothecin-alginate polymer.

The alginate used in the nanospheres in the present disclosure is an FDA (Food and Drug Administration) approved polymer, and the alginate has good biocompatibility, low toxicity, and no antigenicity.

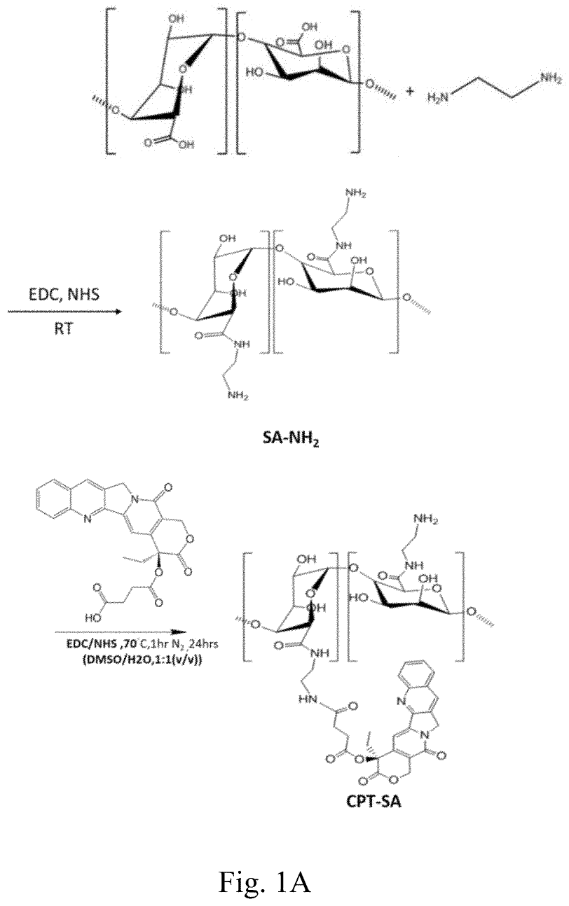

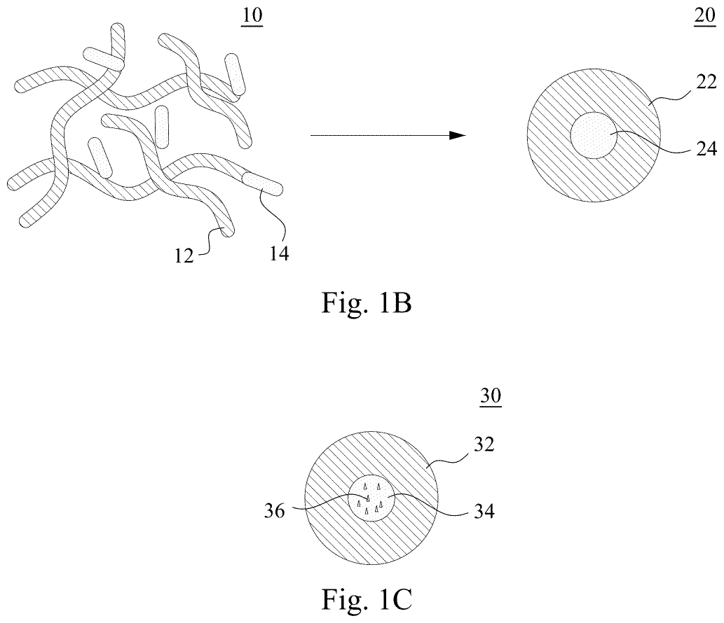

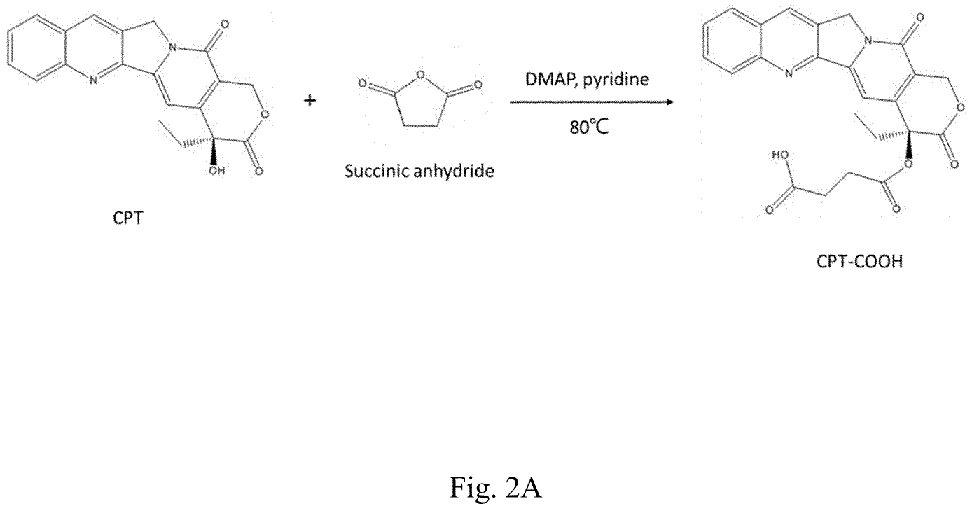

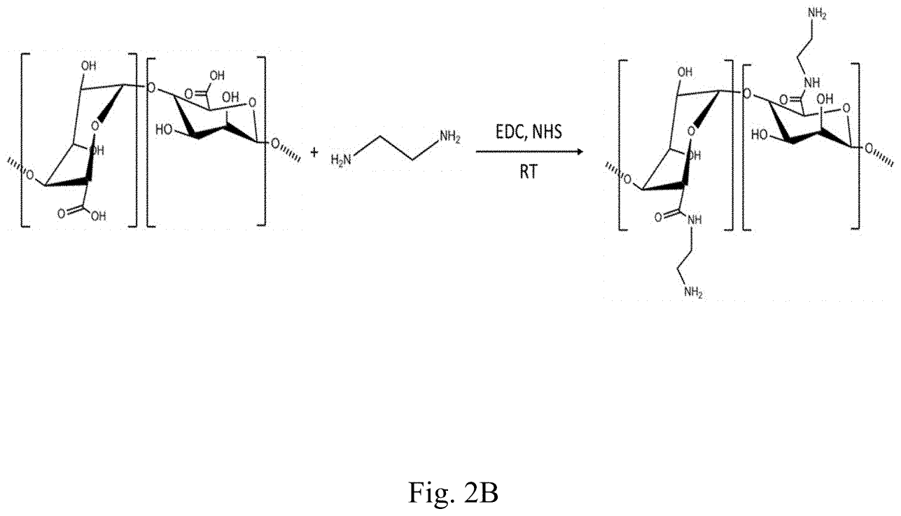

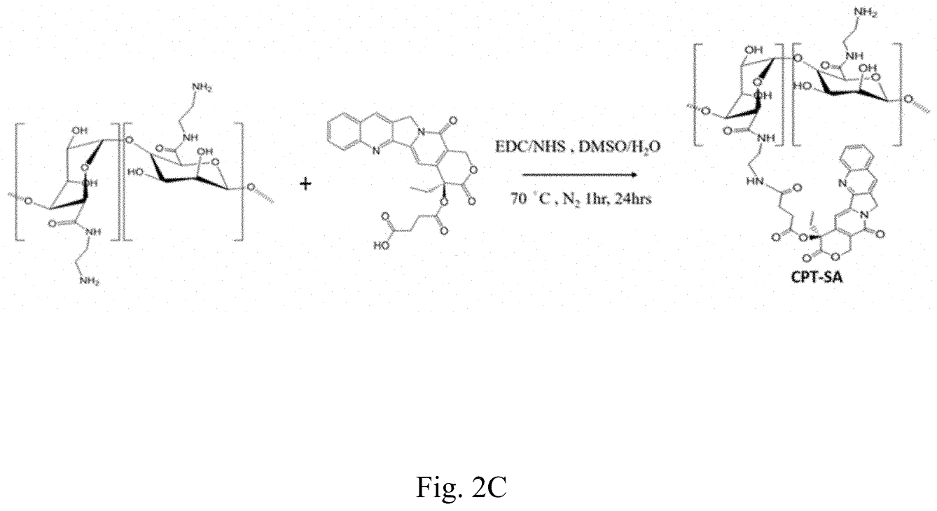

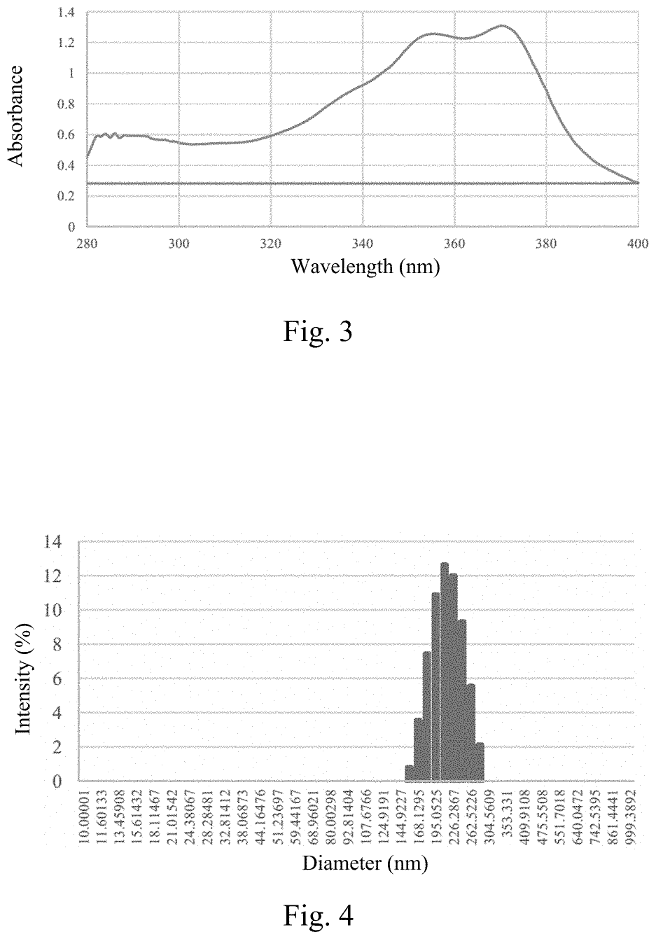

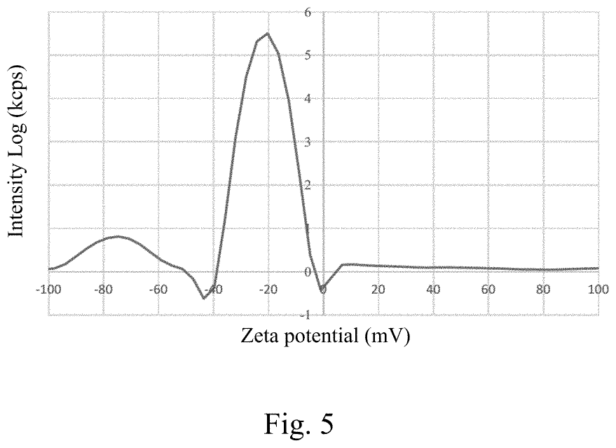

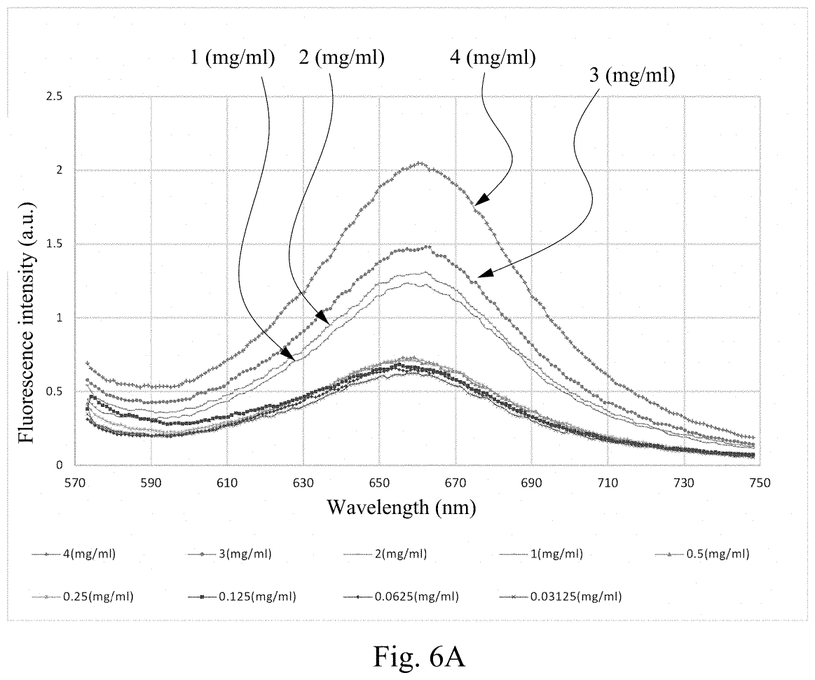

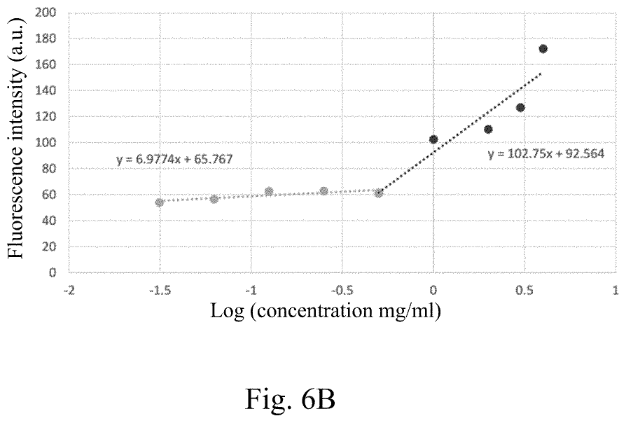

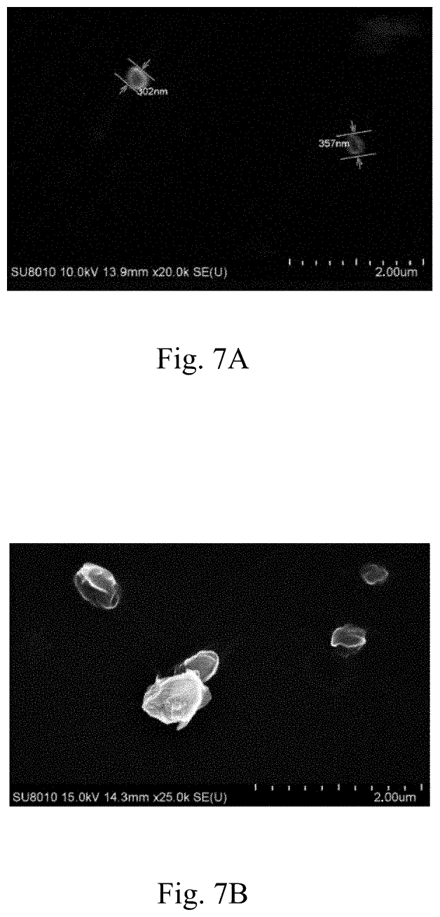

FIG. 1A illustrates a schematic diagram of a method for forming a camptothecin-alginate polymer. Sodium alginate having a low molecular weight reacts with ethylenediamine to form alginate having amine groups (—NH₂). Then, the alginate having amine groups reacts with the camptothecin modified by succinic anhydride (S-CPT) to form a camptothecin-alginate polymer.

The various steps for forming the alginate-camptothecin polymer will be described in more detail in the following disclosure and FIGS. 2A to 2C.

FIG. 1B illustrates a schematic diagram of a self-assembled nanosphere formed from the camptothecin-alginate polymer. A camptothecin-alginate polymer molecule 10 includes alginate 12 and a camptothecin compound 14 grafted onto the alginate 12 The camptothecin-alginate polymer molecule 10 is amphiphilic, the portion of the alginate 12 is hydrophilic, and the portion of the camptothecin compound 14 is hydrophobic. In an aqueous solution, a plurality of camptothecin-alginate polymer molecules 10 aggregate and then self-assemble to form a nanosphere 20. In some embodiments, the nanosphere 20 is a micelle structure having a hydrophilic outer layer 22 and a hydrophobic inner layer 24. The outer layer 22 is substantially composed of the hydrophilic alginate, and the inner layer 24 is substantially composed of the hydrophobic camptothecin compound.

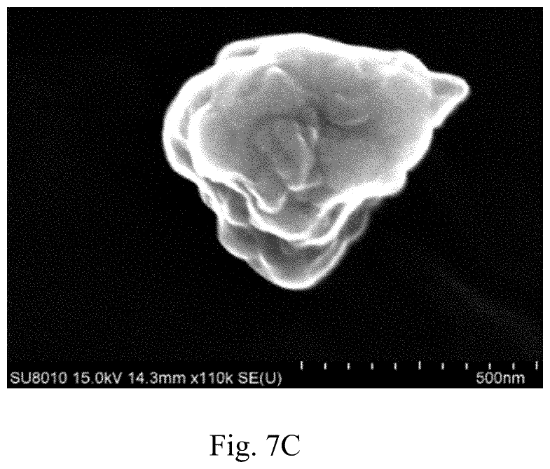

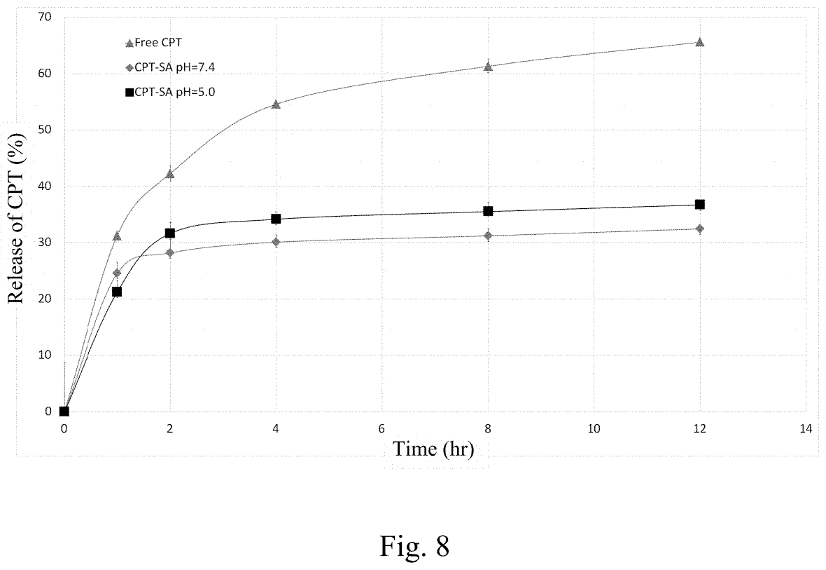

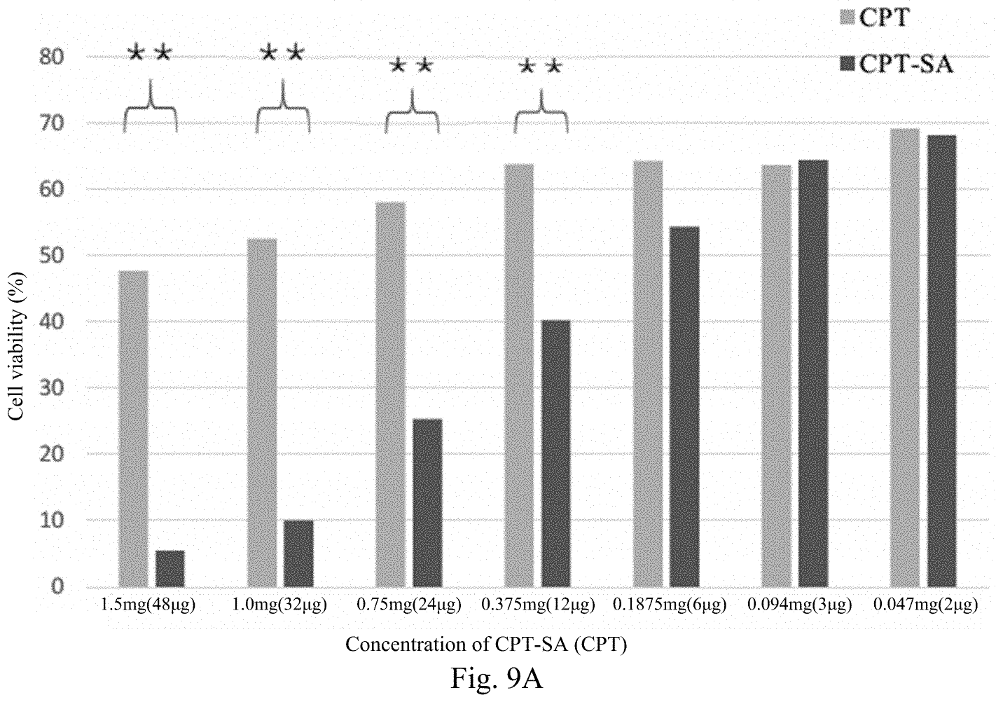

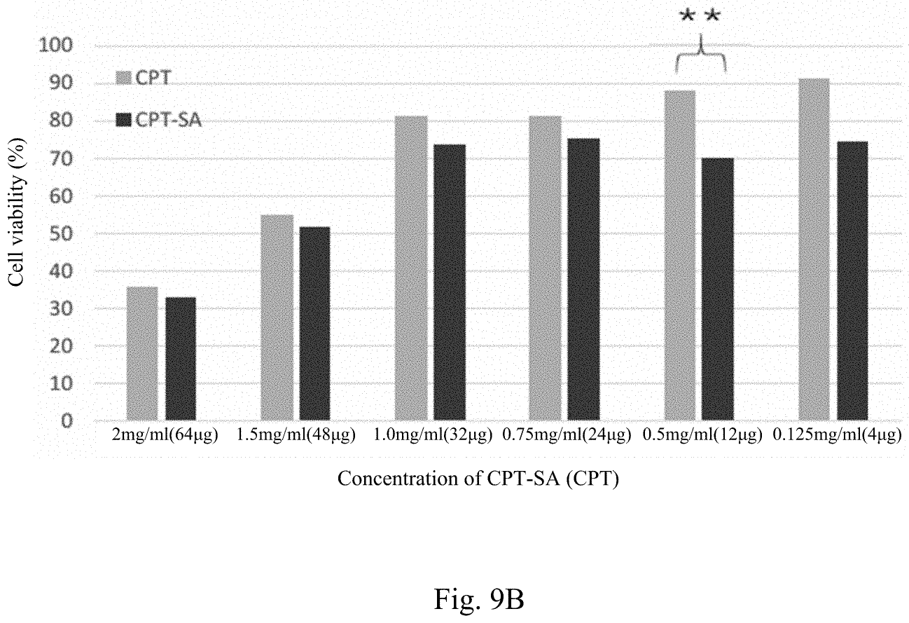

FIG. 10 illustrates a schematic diagram of a nanosphere according to other embodiments of the present disclosure. A nanosphere 30 is a micelle comprising a hydrophilic outer layer 32 and a hydrophobic inner layer 34. The nanosphere 30 further comprises a hydrophobic compound 36 dissolved in the inner layer 34 composed of the camptothecin compound. In other words, the nanosphere 30 not only contains the camptothecin compound, but also can be loaded with other hydrophobic molecules, such as other anti-cancer drugs, contrast agents, or a combination thereof. In some embodiments, other anti-cancer drugs may be an insoluble anti-cancer drug, for example, but not limited to, Paclitaxel, Docetaxel, Adriamycin, Curcumin, Mitoxantrone, Daunorubicin, Etoposide, Teniposide, Vincristine, etc. In some embodiments, the contrast agent may be a hydrophobic contrast agent such as indocyanine green, gadolinium-containing contrast agent, or the like. The hydrophobic molecules can be dissolved in the hydrophobic inner layer of the nanospheres by mixing the hydrophobic molecules with the camptothecin-alginate polymer, e.g., by stirring with a blender or an ultrasound device.

Preparation of the camptothecin-alginate polymer:

Step: degradation of sodium alginate (SA)

The benefit of degrading sodium alginate into low-molecular-weight sodium alginate is that when the drug and the carrier enter a human body, the kidneys can metabolize alginate having a lower molecular weight (for example, a molecular weight between 15,000 and 40,000 Da).

In some embodiments, the molecular weight of the low-molecular-weight sodium alginate is less than about 40,000 Da, such as about 15,000 Da to about 38,000 Da, for example, about, 16,000 Da, about 20,000 Da, about 25,000 Da, about 30,000 Da, about 35,000 Da, or about 38,000 Da.

### Example 1

The sodium alginate is degraded by hydrolysis. 5 g of sodium alginate was dissolved in 45 mL of 1M acetic acid; the temperature was controlled by a heating pack at 85° C.; the reaction solution was stirred for 24 hours. After the reaction, the temperature of the reaction solution was cooled to room temperature, and 5 M sodium hydroxide was added to the reaction solution for neutralization. The reaction solution was dialyzed with ultrapure water for 2 days to remove small molecular impurities. Then the reaction solution was centrifuged at 9,000 rpm for 15 minutes. Afterwards, the upper layer of the solution was taken. Then the upper layer of the solution was frozen and freeze-dried.

Step: esterification reaction of camptothecin (synthesis of CPT-COOH)

FIG. 2A illustrates a step for forming camptothecin having a carboxyl group (—COOH).

### Example 2

0.0352 g camptothecin (CPT) and 0.2024 g succinic anhydrate were taken as reactants, and 0.0122 g 4-Dimethylaminopyridine (DMAP) was taken as a catalyst. 5 mL pyridine was added to serve as a solvent and a reaction catalyst for activating anhydride. The reaction solution was placed in an oil bath with the temperature controlled at 80° C. and stirred continuously, and N₂ gas was introduced for 1 hour. Then, the reaction solution continued to be placed in an oil bath with the temperature controlled at 80° C. and was stirred continuously for 72 hours. After the reaction, the solvent (pyridine) was removed by an oil pump. Then 0.5 mL of 1M HCl was added. Then, water was added, and the reaction solution was centrifuged at 11,000 rpm for 10 minutes. The centrifuge is equipped with Eppendorf FA-45-6-30 rotor having a diameter of 12.3 cm. Then, the upper layer of the solution was poured off. CH₃OH (methanol) was added and refluxed for 1 hour to dissolve the unreacted camptothecin by methanol. The reaction solution was placed at room temperature to recrystallize the product. The camptothecin having a carboxyl group was obtained by suction filtration or centrifugation (at 9,000 rpm for 10 minutes); the upper layer of the solution was poured off, and this step was repeated twice. Then the product was dried by a pump.

Step: modification of sodium alginate with ethylenediamine (synthesizing SA-NH₂)

FIG. 2B illustrates a step for forming alginate having amine groups (—NH₂).

### Example 3

0.25 g of degraded sodium alginate (SA) was dissolved in 15 mL of water, and then 0.48 mL of ethylenediamine, 47.9 mL of 1-Ethyl-3-(3-dimethylaminopropyl) carbodiimide (EDC), 14.3 mg of NHS (N-Hydroxysuccinimide) were added; the reaction solution was stirred at room temperature for 24 hours. Then the reaction solution was dialyzed with ultrapure water; then the reaction solution was centrifuged at 9,000 rpm for 10 minutes, and the upper layer of the solution was taken. Then freeze-drying was performed, and the product obtained was alginate having amine groups.

Step: grafting of the camptothecin having a carboxyl group onto the alginate having amine groups

FIG. 2C illustrates a step for forming camptothecin-alginate polymer from the camptothecin having a carboxyl group and the alginate having amine groups.

### Example 4

19.2 mg of EDC, 8.22 mg of NHS, 20 mg of the camptothecin having a carboxyl group (CPT-COOH) were added to a sample bottle, and the mixture was shaken for 10 minutes; then 20 mg of the sodium alginate having amine groups (SA-NH₂) was added, and 2.5 mL of DMSO (it was used for dissolving CPT-COOH) and 2.5 mL of ultrapure water were added to serve as solvents; then the reaction solution was placed in an oil bath with the temperature was controlled at 70° C. and was continuously stirred, and N₂ gas was introduced for 1 hour. Then the reaction solution continued to be placed in the oil bath with the temperature controlled at 70° C. and was continuously stirred for 24 hours. The reaction solution was dialyzed with ultrapure water to remove by-products; the reaction solution was freeze-dried to obtain the product, i.e., the camptothecin-alginate polymer (CPT-SA).

Because the alginate has amine groups, and the camptothecin has a carboxyl group, the linkage segment between the camptothecin and the alginate in the formed camptothecin-alginate polymer comprises an amide group. As shown in FIGS. 1A and 2C, the camptothecin is grafted onto the alginate through

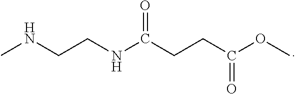

In some embodiments, camptothecin derivatives are used to form camptothecin-alginate polymers. The camptothecin derivatives may be, for example, Topotecan (also known as Hycamtin Irinotecan (also known as Camptosar®), or SN-38. The structural formula of Topotecan is:

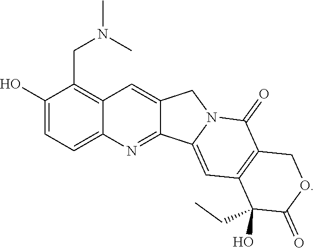 [SMILES: CC[C@@]1(O)C(=O)OCc2c1cc1n(c2=O)Cc2cc3c(CN(C)C)c(O)ccc3nc2-1]

The structural formula of irinotecan is:

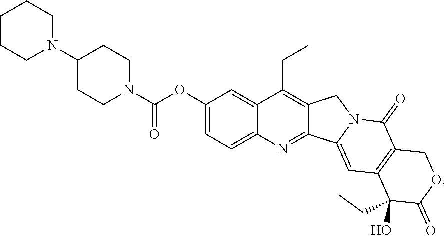 [SMILES: CCc1c2c(nc3ccc(OC(=O)N4CCC(N5CCCCC5)CC4)cc13)-c1cc3c(c(=O)n1C2)COC(=O)[C@]3(O)CC]

The structural formula of SN-38 is:

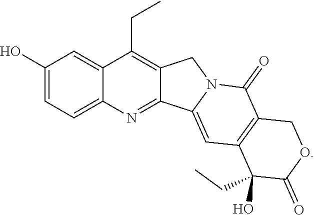 [SMILES: CCc1c2c(nc3ccc(O)cc13)-c1cc3c(c(=O)n1C2)COC(=O)[C@]3(O)CC]

The ICPAC name of SN-38 is (4S)-4,11-Diethyl-4,9-dihydroxy-1,4-dihydro-3H,14H-pyrano[3′,4′:6,7]indolizino[1, 2-b]quinoline-3,14-dione.

After the camptothecin compound was grafted onto the aminated alginate, the chemical properties of the camptothecin compound can be analyzed by ¹H-NMR or UV-VIS to confirm that the camptothecin compound is bonded to the alginate. The camptothecin-alginate polymer is an amphiphilic polymer and has a self-assembly property in aqueous solution. Afterwards, the drug grafting ratio, the particle diameter, and the zeta potential of the nanospheres were further detected, and the morphological characteristics of the nanospheres were observed by electron microscope.

In some examples, the drug grafting ratio (%) of camptothecin in the nanospheres was calculated. As shown in FIG. 3, the absorption spectra of the carboxylated camptothecin (CPT-COOH) in the self-assembled nanospheres were measured by a UV-VIS spectrophotometer. According to the peak value of the CPT-COOH absorption spectrum (the peak value of wavelength 362 nm), the relationship between the absorbance and the concentration of camptothecin having a carboxyl group was drawn. According to Beer-Lambert Law, for the same sample, the light path length and absorption coefficient are the same, and the absorbance of the solution is proportional to the concentration of light-absorbing substances in the solution. Then, by comparing the intensity with the absorption peak of camptothecin-alginate polymer (CPT-SA), and taking the data into the calibration line formula, it was calculated that 3.2 weight % of camptothecin having a carboxyl group was grafted in CPT-SA.

$$Grafting ratio = \frac{weight of the drug ( mg )}{weight of the polymer ( mg )} \times 100 \%$$

FIG. 4 shows the particle diameter size distribution of the nanospheres obtained according to an example. By dynamic light scattering (DLS), it can be found that after the alginate was grafted with camptothecin, the structure formed had the characteristics of nanoparticles. FIG. 5 shows the zeta potential of the nanospheres obtained in this example. Table 1 below shows the size and the zeta potential of the nanoparticles. It can be seen that in this example, the average diameter of the nanoparticles was about 216±29.3 nm, and the zeta potential increased to about −18.22 mV, showing the characteristics of electronegativity.

TABLE 1

| | Name of sample | |
|---|---|---|
| | Size (nm) | Zeta potential (mV) |
| Camptothecin-alginate | 216.0 ± 29.3 | −18.45 mV |
| polymer (0.5 mg/mL) | | |

Measurement of the critical micelle concentration

In another example, using Nile Red as a probe, the hydrophobic Nile Red dye and the nanospheres of the camptothecin-alginate polymer were mixed and stirred, and then the fluorescence intensity of the Nile Red in the nanospheres was measured. Accordingly, the critical micelle concentration can be detected.

FIG. 6A shows the fluorescence intensity values (unit: absorption unit, a.u.) of Nile red in different camptothecin-alginate polymer samples with different concentrations (mg/mL). It shows that when the aqueous solution contained a lower concentration of camptothecin-alginate polymer, the fluorescence intensity of Nile Red measured in the sample was extremely low, which means that there was no or almost no micelle formation. When the aqueous solution contained a higher concentration of camptothecin-alginate polymer, significant Nile red fluorescence can be detected. For example, the concentrations of camptothecin-alginate polymer were 4 mg/mL, 3 mg/mL, 2 mg/mL, and 1 mg/mL. This means that the nanospheres formed by the self-assembly of camptothecin-alginate polymer had micelle structures and the Nile Red entered and was dissolved in the hydrophobic inner layer of the nanospheres.

FIG. 6B shows the relationship between the Log value of the concentration of the camptothecin-alginate polymer and the fluorescence intensity according to the example of FIG. 6A. It can be seen that the camptothecin-alginate polymer had a critical micelle concentration at a concentration of about 0.052 weight %.

FIGS. 7A to 7C show the electron microscope images of the nanospheres according to some embodiments.

FIG. 7A is a transmission electron microscope image of the nanospheres, which shows that the particle diameters of nanospheres are 302 nm and 357 nm, respectively, and the structure of the nanospheres has an outer layer and an inner layer.

FIG. 7B shows a scanning electron microscope image of the nanospheres, which shows that the particle diameter of the nanoparticles obtained in this example ranges between about 200 and about 600 nm.

FIG. 7C shows a scanning electron microscope image of a nanosphere with a particle diameter of about 599 nm.

In Vitro Release of Camptothecin

Afterwards, the drug release characteristics of the nanospheres formed by self-assembly of the camptothecin-alginate polymer were tested. The in vitro release of camptothecin under different conditions were compared; the conditions were: (1) 32 μg/mL camptothecin in phosphate buffered saline (PBS) pH=7.4 (control group); (2) 1 mg/mL camptothecin-alginate polymer in PBS pH=7.4; (3) 1 mg/mL camptothecin-alginate polymer in PBS pH=5.0. The test duration time was 168 hours, the test temperature was 37° C., and the dialysis bag with molecular weight cut-off (MWCO) of 2,000 Da was used.

FIG. 8 shows the release ratios of camptothecin over time under the above-mentioned three conditions. Table 2 below shows the values of the correlation coefficient obtained by substituting the release data measured by in vitro release of drug into Korsmeyer-Peppas model.

TABLE 2

| | System | | |
|---|---|---|---|
| | R² | n | K |
| Camptothecin | 0.957913 | 0.099854 | 44.25132 |
| Camptothecin-alginate | 0.993384 | 0.051399 | 27.19485 |
| polymer (pH = 5.0) | | | |
| Camptothecin-alginate | 0.979796 | 0.068695 | 28.68048 |
| polymer (pH = 7.4) | | | |

As shown in FIG. 8 and Table 2, the camptothecin-alginate polymer released camptothecin in a slower manner than the unmodified camptothecin (the control group, only contains camptothecin) and therefore had a relatively stable property. In addition, the drug release rate of camptothecin was faster in PBS pH=5 than in PBS pH=7.4. These results show that the nanospheres of the camptothecin-alginate polymer could release camptothecin rapidly in a more acidic environment.

Cytotoxicity Test

Afterwards, the cytotoxic effects of the unmodified camptothecin and the camptothecin-alginate polymer on cancer cell lines were compared.

In an example, camptothecin and the camptothecin-alginate polymer were applied to A549 cells (i.e., a human non-small cell lung adenocarcinoma cell line) respectively. The amount of the cells in each test well was 100 cells. 24 hours after the drug administration, the cell counts were measured. FIG. 9A shows the results of the A549 cell test, wherein the horizontal axis represents different drug doses, such as 1.5 mg (48 μg), which represents the addition amount of camptothecin-alginate polymer was 1.5 mg, in which the amount of camptothecin was 48 μg, and the addition amount of the unmodified camptothecin was 48 μg. FIG. 9A shows that at lower concentration, the camptothecin-alginate polymer had significant cytotoxic effects compared with unmodified camptothecin.

Table 3 below shows the difference between the IC₅₀ (i.e., half-maximal inhibitory concentration) value of camptothecin and the IC₅₀ value of the camptothecin-alginate polymer for A549 cells. IC₅₀ decrease factor=[IC₅₀ value of camptothecin]/[IC₅₀ value of the camptothecin-alginate polymer)]. Table 3 shows that the IC₅₀ of camptothecin was 3.87 times the IC₅₀ of the camptothecin-alginate polymer.

TABLE 3
Drug samples appplied to A549 cells

| | IC₅₀ value |
|---|---|
| Camptothecin | 40.1247 μg/mL |
| Camptothecin-alginate polymer | 0.324 mg/mL (10.368 μg/mL) |
| IC₅₀ decrease factor | 3.87 times |

In another example, camptothecin and the camptothecin-alginate polymer were applied to HT-29 cells (i.e., a human colorectal cancer cell line) respectively, wherein the cell amount per test well was 100 cells; 24 hours after the drug was applied, the cell counts were measured. FIG. 9B shows the results of the HT-29 cell test. The horizontal axis represents different drug doses, such as 2 mg (64 μg), which represents the addition amount of the camptothecin-alginate polymer was 2 mg, in which the amount of camptothecin was 64 μg, and the amount of unmodified camptothecin was 64 μg. FIG. 9B shows that at a higher concentration (e.g. 0.5 mg/mL (12 μg)), the camptothecin-alginate polymer had significant cytotoxic effects compared with camptothecin.

Table 4 below shows the difference between the IC₅₀ (i.e., half-maximal inhibitory concentration) value of the camptothecin and the IC₅₀ value of the camptothecin-alginate polymer for HT-29 cells. Table 4 shows that the IC₅₀ of camptothecin was 1.15 times the IC₅₀ of camptothecin-alginate polymer.

TABLE 4
Drug samples applied to Ht-29 cells

| | IC₅₀ value. |
|---|---|
| Camptothecin | 52.34 g /mL |
| Camptothecin-alginate polymer | 1.42 mg/mL (45.44 μg/mL) |
| IC₅₀ decrease factor | 1.15 times |

The method of modifying alginate provided by the present disclosure makes alginate have greater modification potential (i.e., the alginate has —NH₂) and can increase the water solubility and anti-cancer ability of camptothecin. In addition, the camptothecin alginate polymer has the properties of slow-release pharmaceuticals and more potent toxicity against cancer cells under normal physiological buffer conditions, which makes the camptothecin alginate polymer a promising nanodrug delivery system.

In some embodiments, the particle diameter of the nanodrug particles formed by the camptothecin-alginate polymer ranges from about 200 nm to about 600 nm.

In the conventional technology, there is no drug carrier in which camptothecin is grafted onto alginate, and the preparation methods for grafting camptothecin onto other carrier molecules are very tedious and complicated and require multiple steps to form the drug carriers loaded with camptothecin. The present disclosure provides simplified synthesis steps to form nanodrug particles.

Some embodiments of the present disclosure provide the use of the nanodrug particle comprising a camptothecin compound as described above in the manufacture of cancer drugs. In some embodiments, the nanodrug particle can be applied to cancer treatment, and the cancer may be, but not limited to gastric cancer, ovarian cancer, uterine cancer, small cell lung cancer, non-small cell lung cancer, pancreatic cancer, esophageal cancer, oral cancer, rectal cancer, colon cancer, colorectal cancer, renal cancer, prostate cancer, melanoma, liver cancer, gallbladder cancer and other biliary tract cancers, thyroid cancer, bladder cancer, brain and central nervous system cancer, bone tumor, skin cancer, non-Hodgkin's lymphoma, or leukemia.

Although the present invention has been described in considerable detail with reference to certain embodiments thereof, other embodiments are possible. Therefore, the spirit and scope of the appended claims should not be limited to the description of the embodiments contained herein. It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A nanodrug particle comprising:
alginate; and
a camptothecin compound, grafted onto the alginate;
wherein the alginate and the camptothecin compound self-assemble and form a nanosphere.

2. The nanodrug particle of claim 1, wherein the camptothecin compound is grafted to the alginate through

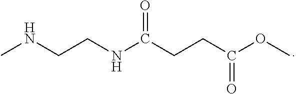

3. The nanodrug particle of claim 1, wherein a linkage segment between the camptothecin compound and the alginate comprises an amide group.

4. The nanodrug particle of claim 1, wherein a molecular weight of the alginate is less than about 40,000 Da.

5. The nanodrug particle of claim 1, wherein the camptothecin compound is selected from the group consisting of camptothecin, Topotecan, Irinotecan, and SN-38.

6. The nanodrug particle of claim 1, wherein a particle diameter of the nanodrug particle ranges from about 200 nm to about 600 nm.

7. The nanodrug particle of claim 1, wherein the nanodrug particle is a micelle, the micelle has an outer portion and an interior portion, the outer portion of the micelle is a hydrophilic layer composed of the alginate, and the interior portion of the micelle is a hydrophobic layer composed of the camptothecin compound.

8. The nanodrug particle of claim 7, wherein the nanodrug particle further comprises a hydrophobic molecule dissolved in the hydrophobic layer of the micelle.

9. A method for treating cancer, comprising:
administering a nanodrug particle to a cancer patient, wherein the nanodrug particle comprises alginate and a camptothecin compound, the camptothecin compound is grafted onto the alginate, and the alginate and the camptothecin compound self-assemble and form a nanosphere.

10. The method for treating cancer of claim 9, wherein the camptothecin compound is grafted to the alginate through

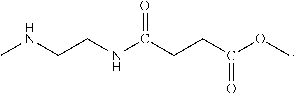

11. The method for treating cancer of claim 9, wherein a linkage segment between the camptothecin compound and the alginate comprises an amide group.

12. The method for treating cancer of claim 9, wherein a molecular weight of the alginate is less than about 40,000 Da.

13. The method for treating cancer of claim 9, wherein the camptothecin compound is selected from the group consisting of camptothecin, Topotecan, Irinotecan, and SN-38.

14. A method for preparing a nanodrug particle, comprising:
modifying alginate to form alginate having amine groups;
modifying a camptothecin compound to form a camptothecin compound having a carboxyl group; and
reacting the alginate having the amine groups and the camptothecin compound having the carboxyl group to form a camptothecin-alginate polymer, wherein the camptothecin-alginate polymer self assembles in an aqueous solution and forms a nanosphere.

15. The method for preparing the nanodrug particle of claim 14, further comprising:
before the modifying the alginate, degrading the alginate until a molecular weight of the alginate is less than about 40,000 Da.

16. The method for preparing the nanodrug particle of claim 14, wherein the modifying the alginate comprises using ethylenediamine as a reactant.

17. The method for preparing the nanodrug particle of claim 14, wherein the modifying the camptothecin compound comprises using succinic anhydride as a reactant.

18. The method for preparing the nanodrug particle of claim 14, wherein the camptothecin compound is selected from the group consisting of camptothecin, Topotecan, Irinotecan, and SN-38.

19. The method for preparing the nanodrug particle of claim 14, further comprising:
adding a hydrophobic compound; and mixing the hydrophobic compound with the nanosphere to dissolve the hydrophobic compound in an interior portion of the nanosphere.

20. The method for preparing the nanodrug particle of claim 14, wherein a particle diameter of the nanosphere ranges from about 200 nm to about 600 nm.
